(19) United States
(12) Patent Application Publication
(10) Pub. No.: US 2004/0063780 A1
(43) Pub. Date: Apr. 1, 2004

(54) Pharmaceutical preparations for treatment of type II diabetes and methods for treatment of type II diabetes

(75) Inventors: Hiroharu Matsuoka, Gotenba-shi (JP); Naoki Taka, Gotenba-shi (JP); Hirotaka Kashiwagi, Gotenba-shi (JP); Kazuharu Ozawa, Gotenba-shi (JP)

Correspondence Address: MORGAN LEWIS & BOCKIUS LLP, 1111 PENNSYLVANIA AVENUE NW, WASHINGTON, DC, 20004, US

(73) Assignee: CHUGAI SEIYAKU KABUSHIKI KAISHA

(21) Appl. No.: 10/284,491

(22) Filed: Oct. 31, 2002

(30) Foreign Application Priority Data
Oct. 1, 2002 (JP) P2002-289101

(51) Int. Cl. A61K 31/34

(52) U.S. Cl. 514/471

(57) ABSTRACT

Pharmaceutical preparations for type II diabetes comprising as active ingredients thereof biguanide derivatives represented by the following general formula (1):

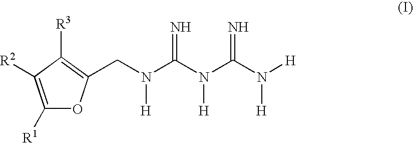

(where R¹, R² and R³ are the same or different and each represents one selected from the group consisting of hydrogen, optionally substituted lower alkyl groups and optionally substituted lower alkylthio groups), or their salts.

### BACKGROUND OF THE INVENTION

1. Field of the Invention

The present invention relates to pharmaceutical preparations for treatment of type II diabetes and to methods for treatment of type II diabetes, and more specifically, it relates to pharmaceutical preparations comprising biguanide derivatives or their salts as active ingredients and to methods for treatment of type II diabetes using them.

2. Related Background Art

Type II diabetes, also known as non-insulin dependent diabetes, is a disease whose onset and progression is caused mainly by impaired insulin secretion (inhibited insulin secretion) and progressive increase in insulin resistance. Impaired insulin secretion produces a quantitative lack of insulin, resulting in blood sugar level increase. On the other hand, progressive increase in insulin resistance is usually compensated for by increased insulin secretion. However, when the limit of insulin secretion increase is reached, there results a relative insulin deficiency, also producing a blood sugar level increase. Onset and progression of type II diabetes is related to both impaired insulin secretion and insulin resistance, and the degree of connection is known to differ from patient to patient.

Insulin sensitivity enhancers exhibit effects of improving insulin resistance and are therefore effective agents for type II diabetes in which insulin resistance is implicated. In glucose tolerance impaired individuals, which are believed to border between healthy individuals and type II diabetics, improvement in insulin resistance is considered effective for preventing onset of diabetes, and insulin sensitivity enhancers are therefore promising for this purpose. Also, in light of mounting evidence for a close connection between progressive insulin resistance and large artery obstruction including myocardial infarction and cerebral apoplexy, insulin sensitivity enhancers are expected to be effective for prevention of these diseases as well (Saishin Igaku Vol.57, No.8, 1739-1746(2002)).

Thiazolidine-based agents such as troglitazone and pioglitazone are known as insulin sensitivity enhancers. However, troglitazone has been removed from the market because it causes serious hepatopathies such as fulminant hepatitis, as a side-effect. Pioglitazone is currently used in the clinic as a treatment for type II diabetes, but is associated with side-effects such as edema and cardiac failure.

Metformin, a type of biguanide-based agent, is also known to have an insulin sensitivity-enhancing effect (N. Engl. J. Med., 338, 867-872(1998)). However, known biguanide agents such as metformin are recognized as being implicated in eliciting lactic acidosis, and because of this risk of lactic acidosis its use is therefore contraindicated for diabetes patients with lactic acidosis anamnesis, impaired renal function, impaired hepatic function, impaired cardiovascular, impaired pulmonary function, tendency to hypoxemia, excessive alcohol intake or gastrointestinal injury, as well as diabetes patients of advanced age (Drugs in Japan: Ethical Drugs, Japan Pharmaceutical Information Center, 2002 (25th edition), p.2170, 2001). Moreover, metformin has a problem that it must be administered in large doses because of its inadequate insulin sensitivity-enhancing effect.

Numerous biguanide derivatives other than metformin are also known, and J. Am. Chem. Soc., 81, 3728-3736 (1959), for example, lists a variety of biguanide derivatives. However, the document merely examines the hypoglycemic action of several biguanide derivatives including metformin in subcutaneous administration tests using guinea pigs with normal blood glucose levels, and does not confirm or describe the presence or degree of insulin sensitivity enhancement or lactic acidosis elicitation.

### SUMMARY OF THE INVENTION

It is an object of the present invention, which has been accomplished in light of the aforementioned problems of the prior art, to provide type II diabetes pharmaceutical preparations with adequate insulin sensitivity-enhancing effects and with low risk of side-effects such as lactic acidosis, as well as methods for treatment of type II diabetes using them.

As a result of avid research directed toward achieving the object stated above, the present inventors have completed the invention upon discovering that biguanide derivatives having a specific structure and their salts exhibit insulin sensitivity-enhancing effects and are effective as pharmaceutical preparations for treatment of type II diabetes.

A pharmaceutical preparation for type II diabetes according to the invention comprises as an active ingredient thereof a biguanide derivative represented by the following general formula (1):

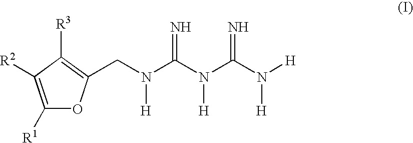

(where R¹, R² and R³ are the same or different and each represents one selected from the group consisting of hydrogen, optionally substituted lower alkyl groups and optionally substituted lower alkylthio groups), or a salt thereof.

A treatment method for type II diabetes according to the invention comprises a step of administering a biguanide derivative represented by general formula (1) above or a salt thereof.

According to the invention, the biguanide derivative represented by general formula (1) is most preferably furfuryl biguanide (where R¹, R² and R³ are all hydrogen).

The biguanide derivatives represented by general formula (1) and their salts exhibit excellent insulin sensitivity-enhancing effects, and the invention is therefore effective for treatment to suppress blood glucose level increase by enhancing insulin sensitivity.

Also according to the invention, the effect of lowering blood glucose levels is preferably exhibited essentially without increase in blood lactic acid levels. This aspect of the invention is useful for providing a treatment to suppress blood glucose level increase without inducing lactic acidosis, and specifically it is useful as a pharmaceutical preparation or treatment method for type II diabetes intended for patients belonging to any one of the group consisting of diabetes patients with lactic acidosis anamnesis, impaired renal function, impaired hepatic function, impaired cardiovascular, impaired pulmonary function, tendency to hypoxemia, excessive alcohol intake or gastrointestinal injury, as well as diabetes patients of advanced age, being particularly useful as a pharmaceutical preparation or treatment method for diabetes patients with impaired renal function.

Since the biguanide derivatives represented by general formula (1) above and their salts exhibit excellent insulin sensitivity-enhancing effects, the invention also relates to (i) any prophylactic agent for type II diabetes comprising as an active ingredient thereof a biguanide derivative represented by general formula (1) or a salt thereof, (ii) any prophylactic agent for large artery obstruction comprising as an active ingredient thereof a biguanide derivative represented by general formula (1) or a salt thereof, (iii) any prophylactic method for type II diabetes comprising a step of administering a biguanide derivative represented by general formula (1) or a salt thereof, and (iv) any prophylactic method for large artery obstruction comprising a step of administering a biguanide derivative represented by general formula (1) or a salt thereof.

The biguanide derivatives represented by the following general formula (1):

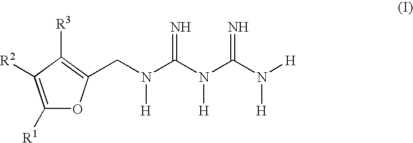

(where R¹, R² and R³ are the same or different and each represents one selected from the group consisting of hydrogen, optionally substituted lower alkyl groups and optionally substituted lower alkylthio groups, except for furfuryl biguanide wherein R¹, R² and R³ are all hydrogen and 1-[(5-methylfuran-2-yl)methyl] biguanide wherein R¹ is methyl and R² and R³ are both hydrogen), are novel compounds, and the invention also relates to these novel biguanide derivatives.

### BRIEF DESCRIPTION OF THE DRAWINGS

FIG. 1 is a graph showing the relationship between blood glucose reduction rate and blood lactic acid level increase rate in an oral glucose tolerance test with administration of furfuryl biguanide or metformin.

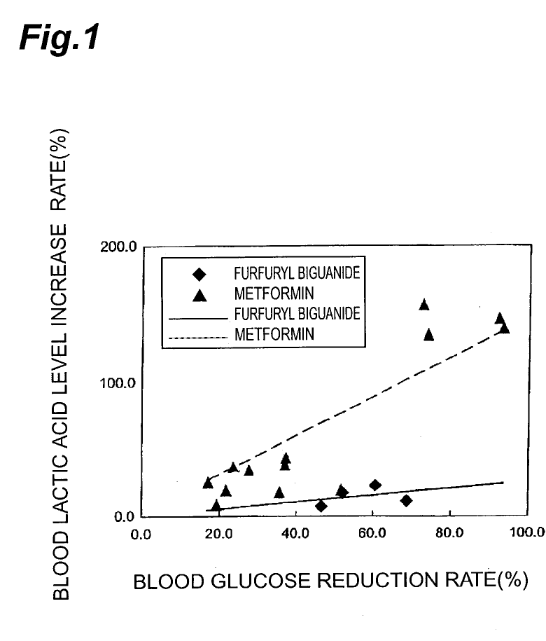

### DESCRIPTION OF THE PREFERRED EMBODIMENTS

Preferred embodiments of the invention will now be explained in detail.

A pharmaceutical preparation for type II diabetes according to the invention comprises as an active ingredient thereof a biguanide derivative represented by the following general formula (1):

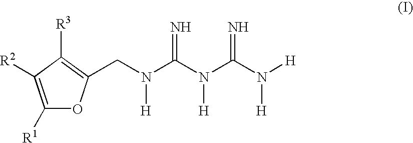

(where R¹, R² and R³ are the same or different and each represents one selected from the group consisting of hydrogen, optionally substituted lower alkyl groups and optionally substituted lower alkylthio groups), or a salt thereof. A prophylactic agent for type II diabetes according to the invention also comprises as an active ingredient thereof a biguanide derivative represented by general formula (1) above or a salt thereof. A prophylactic agent for large artery obstruction according to the invention also comprises as an active ingredient thereof a biguanide derivative represented by general formula (1) above or a salt thereof.

The structural features of the pharmaceutical preparations for type II diabetes of the invention will now be explained.

The biguanide derivatives according to the invention are represented by the following general formula (1):

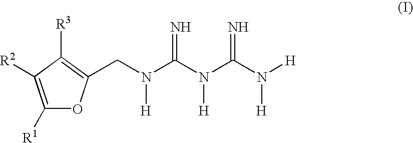

wherein R¹, R² and R³ are the same or different and each represents one selected from the group consisting of hydrogen, optionally substituted lower alkyl groups and optionally substituted lower alkylthio groups. Such biguanide derivatives include the various compounds mentioned below, among which furfuryl biguanide, wherein R¹, R2 and R³ in general formula (1) are all hydrogen, is particularly preferred since it tends to exhibit an adequate insulin sensitivity-enhancing effect without eliciting side-effects such as lactic acidosis.

As the aforementioned lower alkyl groups there are preferred linear or branched alkyl groups of 1-6 carbons, and specifically there may be mentioned methyl, ethyl, n-propyl, isopropyl, n-butyl, isobutyl, sec-butyl, tert-butyl, pentyl groups, hexyl groups and the like. Among these lower alkyl groups, those with 1-5 carbons are preferred, those with 1-4 carbons are more preferred, and methyl is especially preferred.

As the aforementioned lower alkylthio groups there are preferred linear or branched alkylthio groups of 1-6 carbons, and specifically there may be mentioned methylthio, ethylthio, n-propylthio, isopropylthio, n-butylthio, isobutylthio, sec-butylthio, tert-butylthio, pentylthio groups, hexylthio groups and the like. Among these lower alkylthio groups, those with 1-5 carbons are preferred, those with 1-4 carbons are more preferred, and methylthio is especially preferred.

As substituents for the aforementioned lower alkyl groups and alkylthio groups there may be mentioned lower alkylthio and lower alkoxyl groups, among which linear or branched alkylthio groups of 1-6 (more preferably 1-4) carbons are preferred, and methylthio is especially preferred.

The biguanide derivatives represented by the following general formula (1):

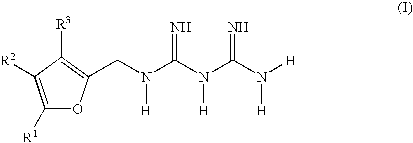

(wherein R¹, R² and R³ are the same or different and each represents one selected from the group consisting of hydrogen, optionally substituted lower alkyl groups and optionally substituted lower alkylthio groups), are novel compounds except for furfuryl biguanide wherein R¹, R² and R³ are all hydrogen and 1-[(5-methylfuran-2-yl)methyl] biguanide wherein R¹ is methyl and R² and R³ are both hydrogen, and they are the biguanide derivatives of the invention. As such novel biguanide derivatives of the invention there may be mentioned specifically 1-[(5-ethylfuran-2-yl)methyl] biguanide, 1-[(5-tert-butylfuran-2-yl)methyl] biguanide, 1-[(4,5-dimethylfuran-2-yl)methyl] biguanide, 1-[(4-methylthiofuran-2-yl)methyl] biguanide, 1-[(5-methylthiomethylfuran-2-yl)methyl] biguanide and 1-[(3-methylthiomethylfuran-2-yl)methyl] biguanide.

The salts of biguanide derivatives represented by general formula (1) above may be in the form of pharmacologically acceptable salts, such as, for example, inorganic acid salts, organic acid salts, acidic amino acid salts and the like. As examples of inorganic acid salts there may be mentioned salts with hydrochloric acid, hydrobromic acid, nitric acid, sulfuric acid and phosphoric acid. As examples of organic acid salts there may be mentioned salts with formic acid, acetic acid, trifluoroacetic acid, fumaric acid, oxalic acid, tartaric acid, maleic acid, citric acid, succinic acid, malic acid, methanesulfonic acid, benzenesulfonic acid and p-toluenesulfonic acid. As examples of acidic amino acid salts there may be mentioned salts with aspartic acid and glutamic acid. Preferred among these salts of biguanide derivatives represented by general formula (1) are salts with inorganic acids, and especially salts with hydrochloric acid.

The aforementioned furfuryl biguanide and 1-[(5-methylfuran-2-yl)methyl] biguanide and their salts may be produced by publicly known methods, and specifically they may be produced by the methods described in U.S. Patent No. 3,821,406, Am. Khim. Zh., 27, 1045-47(1974), Chem. Abstr., 82, 170842m(1975), J. Am. Chem. Soc., 81, 3728-3736(1959), Am. Khim. Zh., 26, 30-34(1973), Chem. Abstr., 78, 159164p(1973), J. Am. Chem. Soc., 81, 3725-3728(1959), J. Chem. Soc., 1063-1069(1946), J. Chem. Soc., 1017-1031(1954), Chem. Abstr., 81, 63361 and elsewhere.

These compounds may be synthesized using furfurylamine as the starting material. The furfurylamine used may be a commercially available product (by Tokyo Kasei Kogyo or Aldrich Chemical, for example). Cyanoguanidine may be mentioned as another starting material, and it may also be used as a commercially available product (by Tokyo Kasei Kogyo, Kanto Kagaku, Wako Pure Chemical or Aldrich Chemical, for example).

The compounds represented by general formula (1) may be synthesized using the corresponding substituted furfurylamines as starting materials. 5-Methylfurfurylamine used may be a commercially available product (by Tokyo Kasei Kogyo or Aldrich Chemical, for example). As other substituted furfurylamines there may be used compounds produced according to the following reaction scheme. The symbols R₁, R² and R³ in the following reaction scheme have the same definition as R¹, R and R³ in general formula (1) above.

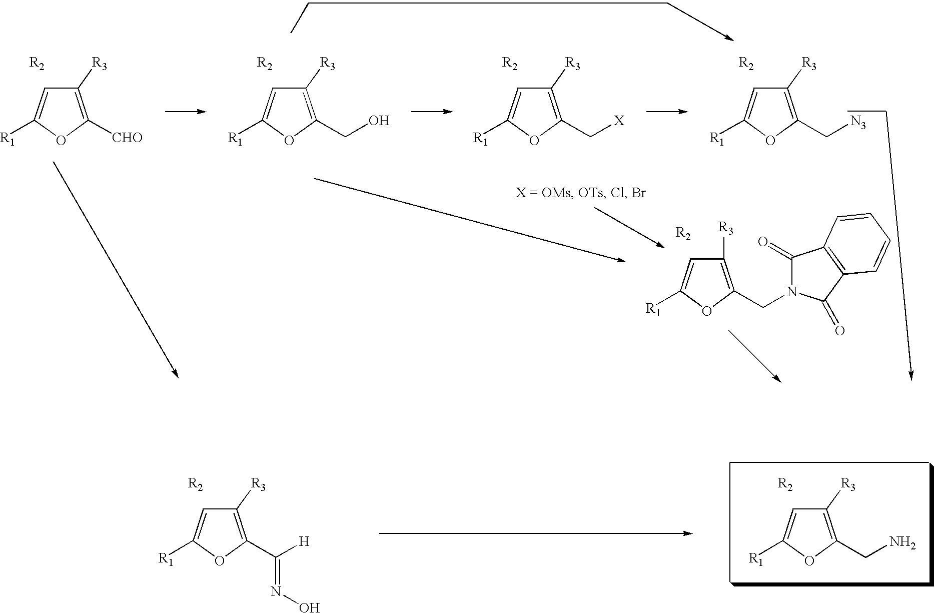

Specifically, the target substituted furfurylamines may be obtained by reducing the corresponding substituted furfuryl azides or substituted furan-2-carbaldehyde oximes (Chem. Pharm. Bull., 39(1), 181-183(1991)). The target substituted furfurylamines may also be synthesized from a substituted furfuryl phthalimides. Substituted furfuryl azides and substituted furfuryl phthalimides may be either directly synthesized from substituted furfuryl alcohols by the Mitsunobu reaction, or they may be synthesized via alkylsulfonic acid esters, typically mesyl or tosyl groups, or via halogenated forms such as chloro or bromo compounds. Substituted furfuryl alcohols may be synthesized by reduction of the corresponding substituted furan-2-carbaldehydes. Substituted furan-2-carbaldehyde oximes may be obtained by reaction of the corresponding substituted furan-2-carbaldehydes and hydroxylamines.

The compounds represented by general formula (1) may be produced by reaction of furfurylamine or substituted furfurylamine with cyanoguanidine in the presence of silylating agents, either in solvents that do not affect the furfurylamine or substituted furfurylamine reaction, or without solvents. As examples of such solvents there may be mentioned hexane, cyclohexane, benzene, toluene, diethyl ether, diisopropyl ether, tert-butylmethyl ether, tetrahydrofuran, dioxane, dichloromethane, 1,2-dichloroethane and chloroform, with dichloromethane, 1,2-dichloroethane, benzene and toluene being preferred. These solvents may also be used in mixed solvents of two or more.

The reaction temperature is not particularly restricted so long as it is a temperature from −78° C. to the boiling point of the reaction mixture, but it is preferably room temperature.

As examples of silylating agents there may be mentioned chlorotrimethylsilane (Me₃SiCl (Me₃Si will hereinafter be abbreviated as TMS)), chlorotriethylsilane (Et₃SiCl), trimethylsilyl trifluoromethanesulfonate (TMSOSO₂CF₃), trimethylsilyl methanesulfonate (TMSOSO₂CH₃), (TMSO)₂SO₂, t-BuMe₂SiOSO₂CF₃, (TMSO) (TMSN)CMe, among which trimethylsilyl trifluoromethanesulfonate and trimethylsilyl methanesulfonate are preferred.

A scheme for the aforementioned production method for the compounds represented by general formula (1) is shown below. The symbols R¹, R2 and R³ in the scheme have the same definition as R¹, R² and R³ in general formula (1) above.

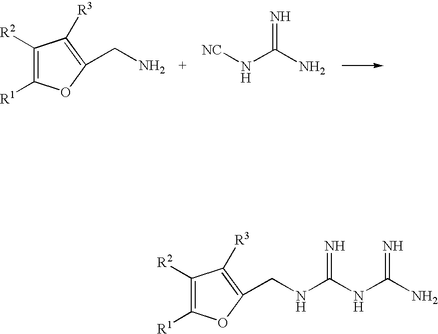

There are no particular restrictions on the specific formulations of the pharmaceutical preparations for treatment of type II diabetes according to the invention, so long as they contain the above-mentioned biguanide derivatives or their salts as active ingredients, and for example, they may be in admixture with additives such as excipients, binders, stabilizers, lubricants, taste correctors, disintegrants, coating agents, coloring agents, buffering agents, aqueous solvents, oily solvents, isotonizing agents, dispersing agents, preservatives, solubilizing agents, fluidizing agents, soothing agents, pH adjustors, antiseptics, bases and the like. Physiologically acceptable carriers may also be used as additives in the pharmaceutical preparations for treatment of type II diabetes.

As examples of excipients there may be mentioned sugars such as lactose, saccharose, glucose, D-mannitol and sorbit, cellulose and its derivatives such as crystalline cellulose, hydroxypropyl cellulose, hydroxypropylmethyl cellulose and methyl cellulose, starches and their derivatives such as corn starch, potato starch, α-starch, dextrin, β-cyclodextrin, carboxymethyl starch sodium and hydroxypropyl starch, silicates such as synthetic aluminum silicate, magnesium aluminosilicate, calcium silicate and magnesium silicate, phosphates such as calcium phosphate, carbonates such as calcium carbonate, sulfates such as calcium sulfate, and tartaric acid, potassium hydrogen tartrate, magnesium hydroxide and the like.

As examples of binders there may be mentioned cellulose and its derivatives such as crystalline cellulose, hydroxypropyl cellulose, hydroxypropylmethyl cellulose and methyl cellulose, starches and their derivatives such as corn starch, potato starch, α-starch, dextrin, β-cyclodextrin, carboxymethyl starch sodium and hydroxypropyl starch, sugars such as lactose, saccharose, glucose, D-mannitol and sorbit, and agar, stearyl alcohol, gelatin, tragacanth, polyvinyl alcohol, polyvinyl pyrrolidone, and the like.

As examples of stabilizers there may be mentioned parahydroxybenzoic acid esters such as methyl paraben and propyl paraben, alcohols such as chlorobutanol, benzyl alcohol and phenylethyl alcohol, phenols such as phenol and cresol, sulfite salts such as sodium bisulfite and sodium sulfite, edetic acid salts such as sodium edetate and tetrasodium edetate, and hydrogenated oils, sesame oil, sodium chondroitin sulfate, dibutyihydroxytoluene, adipic acid, ascorbic acid, stearic L-ascorbate esters, sodium L-ascorbate, L-aspartic acid, sodium L-aspartate, acetyltryptophan sodium, acetanilide, aprotinin solution, aminoethylsulfonic acid, aminoacetic acid, DL-alanine, L-alanine, benzalkonium chloride, sorbic acid and the like.

As examples of lubricants there may be mentioned stearic acids such as stearic acid, calcium stearate and magnesium stearate, waxes such as white beeswax and carnauba wax, sulfates such as sodium sulfate, silicic acid compounds such as magnesium silicate and light silicic anhydride, lauryl sulfates such as sodium lauryl sulfate, and gum arabic powder, cacao butter, carmellose calcium, carmellose sodium, callopeptide, hydrated silicon dioxide, hydrated amorphous silicon oxide, dry aluminum hydroxide gel, glycerin, light liquid paraffin, crystalline cellulose, hydrogenated oil, synthetic aluminum silicate, sesame oil, wheat starch, talc, macrogols, phosphoric acid and the like.

As examples of taste correctors there may be mentioned sugars such as lactose, saccharose, glucose and D-mannitol, and ascorbic acid, L-aspartic acid, sodium L-aspartate, magnesium L-aspartate, aspartame, sweet hydrangea, sweet hydrangea extract, sweet hydrangea powder, aminoethylsulfonic acid, aminoacetic acid, DL-alanine, saccharin sodium, dl-menthol, 1-menthol and the like.

As examples of disintegrants there may be mentioned cellulose and its derivatives such as crystalline cellulose, hydroxypropyl cellulose, hydroxypropylmethyl cellulose and methyl cellulose, carbonates such as calcium carbonate, sodium bicarbonate and magnesium carbonate, starches and their derivatives such as corn starch, potato starch, α-starch, dextrin, β-cyclodextrin, carboxymethyl starch sodium and hydroxypropyl starch, and gelatin, tragacanth, adipic acid, alginic acid, sodium alginate and the like.

As examples of coating agents there may be mentioned cellulose derivatives such as cellulose acetate, hydroxypropyl cellulose, cellulose acetate phthalate and hydroxypropylmethyl cellulose, and shellac, polyvinyl pyrrolidones, polyethylene glycol, macrogols, methacrylic acid copolymers, liquid paraffin, eudragit, and the like.

As examples of coloring agents there may be mentioned indigo carmine, caramel, riboflavin and the like.

As examples of buffering agents there may be mentioned aminoacetic acid, L-arginine, benzoic acid, sodium benzoate, ammonium chloride, potassium chloride, sodium chloride, dried sodium sulfite, dried sodium carbonate, diluted hydrochloric acid, citric acid, calcium citrate, sodium citrate, disodium citrate, calcium gluconate, L-glutamic acid, sodium L-glutamate, creatinine, chlorobutanol, crystalline sodium dihydrogen phosphate, disodium succinate, acetic acid, potassium acetate, sodium acetate, tartaric acid, sodium bicarbonate, sodium carbonate, triethanolamine, lactic acid, sodium lactate solution, glacial acetic acid, boric acid, maleic acid, citric anhydride, anhydrous sodium citrate, anhydrous sodium acetate, anhydrous sodium carbonate, anhydrous sodium hydrogen phosphate, anhydrous trisodium phosphate, anhydrous sodium dihydrogen phosphate, dl-malic acid, phosphoric acid, trisodium phosphate, sodium hydrogen phosphate, dipotassium phosphate, potassium dihydrogen phosphate, sodium dihydrogen phosphate, sodium dihydrogen phosphate hydrate and the like.

As examples of aqueous solvents there may be mentioned distilled water, physiological saline, Ringer's solution and the like.

As examples of oily solvents there may be mentioned vegetable oils such as olive oil, sesame oil, cotton oil and corn oil, and propylene glycol, and the like.

As examples of isotonizing agents there may be mentioned potassium chloride, sodium chloride, glycerin, sodium bromide, D-sorbitol, nicotinamide, glucose, boric acid and the like.

As examples of dispersing agents there may be mentioned stearic acid and its salts such as zinc stearate and magnesium stearate, and gum arabic, propyleneglycol alginate, sorbitan sesquioleate, D-sorbitol, tragacanth, methyl cellulose, aluminum monostearate, aminoalkyl methacrylate copolymer RS, lactose, concentrated glycerin, propylene glycol, macrogols, sodium lauryl sulfate and the like.

As examples of preservatives there may be mentioned alcohols such as chlorobutanol, phenethyl alcohol, propylene glycol and benzyl alcohol, parahydroxybenzoic acid esters such as isobutyl parahydroxybenzoate, ethyl parahydroxybenzoate and methyl parahydroxybenzoate, and benzalkonium chloride, benzethonium chloride, dried sodium sulfite, dried sodium sulfate, cresol, chlorocresol, dibutylhydroxytoluene, potassium sorbate, sodium dehydroacetate, phenol, formalin, phosphoric acid, benzoin, thimerosal, thymol, sodium dehydroacetate and the like.

As examples of solubilizing agents there may be mentioned sodium benzoate, ethylenediamine, citric acid, sodium citrate, glycerin, sodium acetate, sodium salicylate, sorbitan sesquioleate, nicotinamide, glucose, benzyl alcohol, polyvinyl pyrrolidones, acetone, ethanol, isopropanol, D-sorbitol, sodium hydrogen carbonate, sodium carbonate, lactose, urea, saccharose and the like.

As examples of fluidizing agents there may be mentioned stearic acid and its salts such as calcium stearate and magnesium stearate, and hydrated silicon dioxide, talc, absolute ethanol, crystalline cellulose, synthetic aluminum silicate, calcium hydrogen phosphate and the like.

As examples of soothing agents there may be mentioned benzalkonium chloride, procaine hydrochloride, meprylcaine hydrochloride, lidocaine hydrochloride, lidocaine and the like.

As examples of pH adjustors there may be mentioned hydrochloric acid, citric acid, succinic acid, acetic acid, boric acid, maleic acid, sodium hydroxide and the like.

As examples of antiseptics there may be mentioned benzoic acid, sodium benzoate, cetylpyridinium chloride, salicylic acid, sodium salicylate, sorbic acid, potassium sorbate, thymol, methyl parahydroxybenzoate, butyl parahydroxybenzoate and the like.

As examples of bases there may be mentioned vegetable oils such as olive oil, sesame oil and wheat germ oil, and glycerin, stearyl alcohol, polyethylene glycols, propylene glycol, cetanol, lard, white vaseline, paraffin, bentonite, isopropyl lanolin fatty acids, vaseline, polysorbates, macrogols, lauryl alcohol, sodium lauryl sulfate, ethyl linoleate, sodium hydrogen phosphate, rosins and the like.

The amount of a biguanide derivative represented by general formula (1) or its salt in a pharmaceutical preparation for treatment of type II diabetes according to the invention will differ depending on the dosage form, but is preferably from 0.00001-100 wt % with respect to the total of the pharmaceutical preparation for treatment of type II diabetes.

There are no particular restrictions on the dosage form of a pharmaceutical preparation for treatment of type II diabetes according to the invention, and as examples of oral forms there may be mentioned granules, powders, tablets, capsules, syrups, emulsions, suspensions and the like, while as examples of parenteral forms there may be mentioned injections such as subcutaneous injections, intravenous injections, intramuscular injections and intraabdominal injections, percutaneous administration forms such as ointments, creams and lotions, suppository forms such as rectal suppositories and vaginal suppositories, and intranasal administration forms and the like.

The method for producing a pharmaceutical preparation for treatment of type II diabetes according to the invention need only employ a biguanide derivative represented by general formula (1) or its salt to produce a pharmaceutical preparation for treatment of type II diabetes (preferably one which suppresses blood glucose level increase by enhancing insulin sensitivity, and more preferably one which has an effect of lowering blood glucose levels (hypoglycemic effect) essentially without raising blood lactic acid levels). The specific method is not particularly restricted, and the various formulations described above containing biguanide derivatives represented by general formula (1) or their salts may be produced by publicly known methods commonly used in formulating steps. That is, any of various formulations may be obtained by appropriate mixture of a prescribed amount of a biguanide derivative represented by general formula (1) or its salt with the aforementioned additives, depending on the desired dosage form of the pharmaceutical preparation for treatment of type II diabetes.

The “insulin sensitivity-enhancing effect” of the pharmaceutical preparations for treatment of type II diabetes according to the invention will now be explained.

The strength of the insulin sensitivity-enhancing effect may be evaluated based on the degree of blood glucose reduction rate with administration of the drug agent to KKAy mice. KKAy mice are an insulin resistance-exhibiting animal diabetes model (Nihon Rinsho, Vol.60, Special Edition No.8, 38-44(2002)), and it is known that sulfonylurea-based hypoglycemic agents, used as type II diabetes therapeutic agents based on their insulin secretion-promoting effect, are not effective in this animal model (Medical Pharmacy, Vol.24, No.3, 131-136, (1990)). In hypoglycemic tests by oral administration using KKAy mice, approximately 45% suppression of blood glucose level in KKAy mice produces a level similar to healthy mice, and therefore a blood glucose reduction rate of 40-50% is preferred.

Measurement of blood glucose reduction rate in KKAy mice by a hypoglycemic test with oral administration may be carried out by a publicly known method, but the following method is preferred.

Specifically, a group of six 11-week-old male mice (KKAy/Ta) is used for the test. Blood is sampled from the tail for measurement of the blood glucose levels before treatment as a control. After sampling, the biguanide derivative is dissolved in a 0.5% CMC-Na (sodium carboxymethyl cellulose) solution to a suitable concentration and orally administered at a dose of 10 mL/kg. As a control there are prepared mice administered only the solvent. Blood is sampled from the tail to measure the blood glucose levels 1, 2, 4 and 6 hours after administration of the drug. The blood glucose levels are measured using a Glucose CII-Test Wako (Wako Pure Chemical Industries, Ltd.).

The blood glucose reduction rate is calculated according to the following formula.

Blood glucose reduction rate (%)=[(AUC for blood glucose of control group—AUC for blood glucose of compound-administered group)/AUC for blood glucose of control group]×100

The AUC for blood glucose represents the area in a graph of the blood glucose level changes after administration of the drug plotted with respect to time, up to 6 hours after administration with a glucose level of 0 as the baseline. Specifically, the AUC for the blood glucose level may be calculated by the following formula, where A=blood glucose level before drug administration, B=blood glucose level 1 hour after drug administration, C=blood glucose level 2 hours after drug administration, D=blood glucose level 4 hours after drug administration, E=blood glucose level 6 hours after drug administration.

AUC for blood glucose level=1×((A+B)/2)+1×((B+C)/2)+2×((C+D)/2)+2×((D+E)/2)

A blood glucose reduction rate of approximately 45% is a blood glucose reduction rate to a level comparable to that of normal mice.

The strength of the insulin sensitive-enhancing effect may be evaluated by the degree of blood glucose reduction rate upon administration of the drug to db/db mice as an insulin resistance-exhibiting animal diabetes model (Nihon Rinsho, Vol.60, Special Edition No.8, 38-44(2002)). In a glucose tolerance test with oral administration to db/db mice, approximately 50% suppression of blood glucose level in the db/db mice produces a level increase similar to that of healthy mice, and therefore a blood glucose reduction rate of at least 40%, and especially at least 50%, is preferred.

Measurement of blood glucose reduction rate in db/db mice by a glucose tolerance test with oral administration may be carried out by a publicly known method, but the following method is preferred. Specifically, 11- to 17-week-old female mice (C57BLKS/J-m+/+Lepr<db>(db/db) ) are starved for 18-24 hours. A group of five or six mice is used for the test. Blood is sampled from the tail for measurement of the blood glucose levels before treatment as a control. After sampling, the biguanide derivative is dissolved in phosphate-buffered saline to a suitable concentration and subcutaneously administered at a dose of 5 ml/kg. As a control there are prepared mice administered only the solvent. Glucose is then administered orally at a dose of 3 g/6 ml/kg at 30 minutes after administration of the compound or solvent as an oral glucose tolerance test. Blood is sampled from the tail for measurement of the blood glucose levels 30 minutes, 1 hour and 2 hours after glucose administration. The blood glucose levels are measured using a New Blood Sugar Test (Roche Diagnostics) or a Glucose CII-Test Wako (Wako Pure Chemical Industries, Ltd.).

The blood glucose reduction rate is calculated according to the following formula.

Blood glucose reduction rate (%)=[(AUC for blood glucose increase level of solvent-administered group—AUC for blood glucose increase level of compound-administered group)/AUC for blood glucose increase level of solvent-administered group]×100

The AUC for blood glucose increase level represents the area of the increase portion in a graph of the blood glucose level changes after glucose administration plotted with respect to time, up to 2 hours after glucose administration, with the glucose level prior to glucose administration as the baseline. Specifically, the AUC for the blood glucose increase level may be calculated by the following formula, where A=blood glucose level before glucose administration, B=blood glucose level 30 minutes after glucose administration, C=blood glucose level 1 hour after glucose administration, D=blood glucose level 2 hours after glucose administration.

AUC for blood glucose increase level=0.5×((A+B)/2-A)+0.5×((B+C)/2-A)+1×((C+D)/2-A)

The effect of “lowering blood glucose levels essentially without increasing blood lactic acid levels”, as the preferred effect of the pharmaceutical preparation for treatment of type II diabetes according to the invention, will now be explained.

For the purpose of the invention, lowering blood glucose levels essentially without increasing blood lactic acid levels means that when blood glucose reduction rate and blood lactic acid levels are measured by an oral glucose tolerance test, the dosage of the diabetes treatment agent which exhibits a blood glucose reduction rate of 40-60% results in a blood lactic acid level increase rate of preferably no greater than 15%. For example, when blood glucose reduction rate and blood lactic acid levels are measured by the aforementioned oral glucose tolerance test for a typical diabetes patient exhibiting an initial blood lactic acid level of 4-33 mg/dL, even administration of the diabetes treatment agent at a dose which exhibits a blood glucose reduction rate of 40-60% does not increase the blood lactic acid level above 38 mg/dL. Also, at a dose of the pharmaceutical preparation for treatment of type II diabetes which exhibits a blood glucose reduction rate of 60-80%, the blood lactic acid level increase rate is preferably no greater than 35%, more preferably no greater than 30% and most preferably no greater than 25%. For example, when blood glucose reduction rate and blood lactic acid levels are measured by the aforementioned oral glucose tolerance test for a typical diabetes patient exhibiting an initial blood lactic acid level of 4-33 mg/dL, preferably even administration of the pharmaceutical preparation for treatment of type II diabetes at a dose which exhibits a blood glucose reduction rate of 60-80% does not increase the blood lactic acid level above 45 mg/dL.

Measurement of blood glucose reduction rate and blood lactic acid levels by the aforementioned oral glucose tolerance test may be carried out by a publicly known method, and the measurement of the former may be carried out by the method described above, while the following method is preferred for measurement of the latter. Specifically, 11- to 17-week-old female mice (C57BLKS/J-m+/+Lepr<db>(db/db)) are starved for 18-24 hours. A group of five or six mice is used for the test. Blood is sampled from the tail for measurement of the blood lactic acid levels before treatment as a control. After sampling, the biguanide derivative is dissolved in phosphate-buffered saline to a suitable concentration and subcutaneously administered at a dose of 5 ml/kg. As a control there are prepared mice administered only the solvent. Glucose is then administered orally at a dose of 3 g/6 ml/kg at 30 minutes after administration of the compound or solvent as an oral glucose tolerance test. Blood is sampled from the tail for measurement of the blood lactic acid levels 30 minutes, 1 hour and 2 hours after glucose administration. The blood lactic acid levels are measured using an “Asuka Sigma” (Sigma Diagnostics)

The blood lactic acid level increase rate is calculated according to the following formula.

Blood lactic acid level increase rate (%)=[(AUC for blood lactic acid level of compound-administered group—AUC for blood lactic acid level of solvent-administered group)/AUC for blood lactic acid level of solvent-administered group]×100

The AUC for blood lactic acid level represents the area in a graph of the blood lactic acid level changes after glucose administration plotted with respect to time, up to 2 hours after glucose administration. Specifically, the AUC for the blood lactic acid level may be calculated by the following formula, where E=blood lactic acid level before glucose administration, F=blood lactic acid level 30 minutes after glucose administration, G=blood lactic acid level 1 hour after glucose administration, H=blood lactic acid level 2 hours after glucose administration.

AUC for blood lactic acid level=0.5×(E+F)/2+0.5×(F+G)/2+1×(G+H)/2

A treatment and prophylactic method for type II diabetes according to the invention and a prophylactic method for large artery obstruction according to the invention will now be explained. All of these methods of the invention need only include a step of administering a biguanide derivative represented by general formula (1) above or its salt, and the specific route of administration and dosage form are not particularly restricted.

The preferred object of administration will be described first. Biguanide derivatives according to the invention and their salts have the excellent insulin sensitivity-enhancing effect described above, and they preferably lower blood glucose levels essentially without raising blood lactic acid levels. They are therefore useful for treatment to suppress blood glucose level increase which do not induce lactic acidosis, and are effective for administration to diabetes patients and especially to diabetes patients who are prone to lactic acidosis. Diabetes patients prone to lactic acidosis include, for example, diabetes patients with lactic acidosis anamnesis, impaired renal function, impaired hepatic function, impaired cardiovascular, impaired pulmonary function, tendency to hypoxemia, excessive alcohol intake or gastrointestinal injury, as well as diabetes patients of advanced age.

Biguanide derivatives and their salts, or pharmaceutical preparations for treatment of type II diabetes comprising them, according to the invention, are particularly effective for diabetes patients who are prone to lactic acidosis as explained above, and are especially suitable for administration to diabetes patients with impaired renal function. Impaired renal function includes, specifically, for example chronic renal failure, diabetic nephropathy, glomerular nephritis, immune complex nephritis, acute renal failure, interstitial nephritis, renal sclerosis, renal infarction, abnormal tubular function, drug-induced nephropathy, agricultural chemical-induced nephropathy, uremia, and the like.

The method of administering a biguanide derivative or its salt according to the invention is not particularly restricted, and for example, the agent may be administered orally or parenterally as a drug composition (preparation) using the aforementioned additives with a biguanide derivative represented by general formula (1) or its pharmacologically acceptable salt.

The dosage of a biguanide derivative represented by general formula (1) or its salt may be appropriately determined based on the species of subject (human or other warm-blooded animal, for example), the severity of symptoms, the age, route of administration, physician diagnosis, etc., and for an adult, for example, the dosage of a biguanide derivative represented by general formula (1) will be preferably 0.1-2000 mg/kg per day in the case of oral administration, and preferably 0.1-1000 mg/kg per day in the case of parenteral administration. These dosages are the values per unit weight (1 kg) of the subject of administration. According to the invention, the dosage may be administered once during a period of 1-7 days or divided over several times, depending on the severity of symptoms, the physician diagnosis, etc.

By thus administering an effective dose of a biguanide derivative represented by general formula (1) or its salt, it is possible to adequately suppress increase in blood glucose levels by an excellent insulin sensitivity-enhancing effect, and to sufficiently lower blood glucose levels, preferably while adequately inhibiting increase in blood lactic acid levels.

Since the biguanide derivatives represented by general formula (1) and their salts have an excellent insulin sensitivity-enhancing effect as described above, they are useful as active ingredients of pharmaceutical preparations for treatment of type II diabetes which effectively prevent onset of diabetes through enhancement of insulin resistance. Moreover, in light of the close connection between progressive insulin resistance and large artery obstruction including myocardial infarction and cerebral apoplexy, the biguanide derivatives represented by general formula (1) and their salts having such insulin sensitivity-enhancing effects are also useful as active ingredients for prophylactic agents against large artery obstruction.

### EXAMPLES

The present invention will now be explained in greater detail through examples and comparative examples, with the understanding that these examples are not limitative on the invention.

### Synthesis Example 1

### Synthesis of Furfuryl Biguanide

Trimethylsilyl trifluoromethanesulfonate (11.2 ml) was added to a dichloromethane solution (50 ml) of furfurylamine (5.0 g), and the mixture was stirred at room temperature for 30 minutes. Cyanoguanidine (4.33 g) was added to the mixture which was then stirred overnight. The reaction mixture was subjected to amine treatment silica gel column chromatography (methanol:dichloromethane=10:100) to obtain the target substance as an oil (5.85 g). The results of structural analysis of the oily substance were as follows.

¹H-NMR(DMSO-d₆) d: 4.33 (2H, s), 6.32 (1H, d, J=2.97 Hz), 6.40 (1H, s), 6.85 (6H, m), 7.59 (1H, s); Fab-MS: 182 (M+H⁺); HPLC RT: 6.5 min.

The structural formula of the obtained compound is as follows.

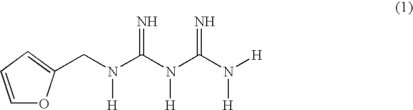

The amine treatment silica gel column chromatography was carried out using Silica Gel Chromatorex NH DM1020 (100 mm particle size) by Fuji Silysia Chemical Ltd. The HPLC apparatus used was an L-6200 by Hitachi Ltd., the HPLC column used was a Develosil ODS HG-5, 4.6×150 mm by Nomura Chemical Co., Ltd, and the HPLC retention time (RT: min) measurement was conducted in the following manner. Specifically, it was conducted under conditions with an aqueous mixture of 10% methanol/0.1 M ammonium acetate as the mobile phase, a flow rate of 1 ml/min and a detection wavelength of 240 nm.

### Synthesis Example 2

### Synthesis of (1-(5-Methylfurfuryl) Biguanide

Trimethylsilyl trifluoromethanesulfonate (5.37 ml) was added to a 1,2-dichloroethane mixture (19 ml) of 5-methylfurfurylamine (3.0 g), and the mixture was stirred at room temperature for 1 hour. Cyanoguanidine (2.27 g) was added to the mixture which was then stirred overnight at room temperature, after which the mixture was heated to reflux for 1.5 hours. The reaction solution was cooled to room temperature and then subjected to amine treatment silica gel column chromatography (methanol:dichloromethane=10:100) to obtain the target substance as a white powder (4.50 g). The results of structural analysis of the white powder substance were as follows.

¹H-NMR(DMSO-d₆) d: 2.24 (3H, s), 4.25 (2H, s), 6.00 (1H, s), 6.18 (1H, m), 6.40-8.40 (6H, m); Fab-MS: 196 (M+H⁺); HPLC RT: 21.7 min.

The structural formula of the obtained compound is as follows.

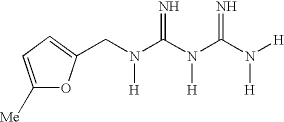

The amine treatment silica gel column chromatography was carried out using Silica Gel Chromatorex NH DM1020 (100 mm particle size) by Fuji Silysia Chemical Ltd. The HPLC apparatus used was an L-6200 by Hitachi Ltd., the HPLC column used was a Develosil ODS HG-5, 4.6×150 mm by Nomura Chemical Co., Ltd, and the HPLC retention time (RT: min) measurement was conducted in the following manner. Specifically, it was conducted under conditions with an aqueous solution of 10% methanol/0.1 M ammonium acetate as the mobile phase, a flow rate of 1 ml/min and a detection wavelength of 240 nm. Fab-MS was measured using a 70-SEQ by VG Analytical Co.

### Synthesis Example 3

### Synthesis of 1-[(5-Ethylfuran-2-yl)methyl] Biguanide

Trimethylsilyl trifluoromethanesulfonate (9.72 mL) was added to a 1,2-dichloroethane solution (32 mL) of (5-ethylfuran-2-yl)methylamine (5.61 g), the mixture was stirred at room temperature for 30 minutes, and cyanoguanidine (3.77 g) was added to the mixture which was then stirred overnight at room temperature. The reaction solution was subjected to amine treatment silica gel column chromatography (methanol:chloroform=10:100) and the solvent was distilled off under reduced pressure to obtain the target substance as an oil (1.48 g). The results of structural analysis of the oily substance were as follows.

1H-NMR(DMSO-d₆) δ: 1.16 (3H, t, J=7.42 Hz), 2.58 (2H, q, J=7.42 Hz), 4.26 (2H, s), 6.00 (1H, brs), 6.19 (1H, d, J=2.47 Hz), 6.54-8.29 (6H, m); MS(ESI⁺): 210[M+1]⁺; HPLC RT: 11.7 min. (mobile phase: 30% methanol).

The structural formula of the obtained compound is as follows.

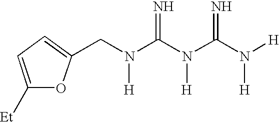

The amine treatment silica gel column chromatography conditions were the same as for Synthesis Example 2, and the HPLC conditions were the same as for Synthesis Example 2 except that the methanol concentration for the mobile phase was as indicated above. LCMS was measured by the ionization method (ESI⁺) using LCQ by Thermo Finnigan Co.

### Synthesis Example 4

### Synthesis of 1-[(5-tert-butylfuran-2-yl)methyl] Biguanide

1) Triphenylphosphine (31.2 g) and phthalimide (17.5 g) were added to a THF solution (240 mL) of (5-tert-butylfuran-2-yl)methyl alcohol (18.3 g), and then diethylazo dicarboxylate (DEAD, 18.7 mL) was added dropwise while cooling on ice, and the mixture was stirred for 2 hours. The solvent of the reaction solution was removed under reduced pressure, diethyl ether was added and the precipitated insoluble portion was filtered off. The solvent of the filtrate was removed under reduced pressure, the obtained residue was subjected to silica gel column chromatography (hexane:dichloromethane=1:1) and the solvent was distilled off under reduced pressure to obtain the target substance N-[(5-tert-butylfuran-2-yl)methyl] phthalimide as a powder (15.6 g). The results of structural analysis of the obtained substance were as follows.

¹H-NMR(CDCl₃) δ: 1.22 (9H, s), 4.81 (2H, s), 5.85 (1H, d, J=3.13 Hz), 6.19 (1H, d, J=3.13 Hz), 7.67-7.87 (4H, m).

2) A 40% methanol solution (70 mL) of methylamine was added to a methanol/dichloromethane mixture (1:2, 105 mL) containing the N-[(5-tert-butylfuran-2-yl)methyl] phthalimide (15.6 g) obtained in 1), and the mixture was stirred overnight at room temperature. The reaction solution was distilled under reduced pressure and then diethyl ether was added to the obtained residue, the precipitated insoluble portion was filtered off and the filtrate was distilled off under reduced pressure to obtain the target substance (5-tert-butylfuran-2-yl)methylamine as an oil (7.91 g) The results of structural analysis of the obtained substance were as follows.

¹H-NMR(CDCl₃) δ: 1.27 (9H, s), 3.77 (2H, s), 5.85 (1H, d, J=2.96 Hz), 5.98 (1H, d, J=2.96 Hz).

3) Trimethylsilyl trifluoromethanesulfonate (4.54 mL) was added to a 1,2-dichloroethane solution (16 mL) containing the (5-tert-butylfuran-2-yl)methylamine (3.50 g) obtained in 2), and the mixture was stirred at room temperature for 30 minutes. Cyanoguanidine (1.92 g) was added and the mixture was heated to reflux for 1 hour. The reaction solution was subjected to amine treatment silica gel column chromatography (methanol:chloroform=10:100) to obtain the target substance as a white powder (1.23 g). The results of structural analysis of the obtained white powder were as follows.

¹H-NMR(DMSO-d₆) δ: 1.23 (9H, s), 4.27 (2H, s), 5.97 (1H, d, J=2.96 Hz), 6.16 (1H, d, J=2.96 Hz), 6.70-8.30 (6H,m); MS(ESI⁺): 238[M+1]⁺HPLC RT: 7.6 min. (mobile phase: 50% methanol).

The structural formula of the obtained compound is as follows.

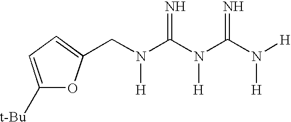

The conditions for amine treatment silica gel column chromatography and LCMS were the same as for Synthesis Example 3, and the HPLC conditions were the same as for Synthesis Example 2 except that the methanol concentration for the mobile phase was as indicated above.

### Synthesis Example 5

### Synthesis of 1-[(4,5-Dimethylfuran-2-yl)methyl] Biguanide

1) Sodium azide (11.3 g) and triphenylphosphine (45.6 g) were added to an N,N-dimethylformamide (DMF) solution (260 mL) of (4,5-dimethylfuran-2-yl)methyl alcohol (16.9 g), after which carbon tetrabromide (57.7 g) was added while cooling on ice and the mixture was stirred at room temperature for 1 hour. The reaction solution was poured into ice water and extracted with diethyl ether, and the organic layer was washed with a saturated aqueous sodium bicarbonate solution and then with saturated saline. After drying the organic layer over anhydrous magnesium sulfate, the solvent was distilled off under reduced pressure. The obtained residue was subjected to silica gel column chromatography (hexane) to obtain the target substance 2-azidomethyl-4,5-dimethylfuran (11.5 g) as an oil. The results of structural analysis of the obtained substance were as follows.

¹H-NMR(CDCl₃) δ: 1.92 (3H, s), 2.20 (3H, s), 4.19 (2H, s), 6.11 (1H, s).

2) Aluminum lithium hydride (1.26 g) was gradually added to a diethyl ether solution (66 mL) containing the 2-azidomethyl-4,5-dimethylfuran obtained in 1) (5.01 g) while cooling on ice, and the mixture was stirred for 30 minutes. The reaction solution was poured into ice water and the obtained suspension was filtered with celite. The filtrate was extracted with diethyl ether, the organic layer was washed with a saturated aqueous sodium bicarbonate solution and dried over anhydrous magnesium sulfate, and then the solvent was distilled off under reduced pressure to obtain the target substance (4,5-dimethylfuran-2-yl)methylamine (2.66 g) as an oil. The results of structural analysis of the obtained substance were as follows.

¹H-NMR(CDCl₃) δ: 1.90 (3H, s), 2.18 (3H, s), 3.71 (2H, s), 5.89 (1H, s).

3) Trimethylsilyl trifluoromethanesulfonate (4.61 mL) was added to a 1,2-dichloroethane solution (22 mL) containing (4,5-dimethylfuran-2-yl)methylamine (2.66 g), and the mixture was stirred at room temperature for 45 minutes, after which cyanoguanidine (1.79 g) was added and the mixture was stirred overnight at room temperature. The reaction solution was subjected to amine treatment silica gel column chromatography (methanol:chloroform=10:100) to obtain the target substance as an oil (3.00 g). The results of structural analysis of the obtained oily substance were as follows.

¹H-NMR(DMSO-d₆) δ: 1.87 (3H, s), 2.15 (3H, s), 4.20 (2H, s), 6.08 (1H, s), 6.50-8.30 (6H, m); MS(ESI⁺): 210[M+1]⁺; HPLC RT: 8.9 min. (mobile phase: 30% methanol);

The structural formula of the obtained compound is as follows.

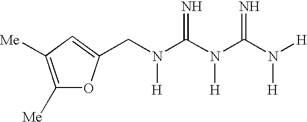

The conditions for amine treatment silica gel column chromatography and LCMS were the same as for Synthesis Example 2, and the HPLC conditions were the same as for Synthesis Example 2 except that the methanol concentration for the mobile phase was as indicated above.

### Synthesis Example 6

### Synthesis of 1-[(4-Methylthiofuran-2-yl)methyl] Biguanide

1) 2 N hydrochloric acid (20 mL) was added to a diethyl ether/methanol mixture (5:1, 120 mL) containing 2-diethoxymethyl-4-methylthiofuran (10.23 g), and the mixture was stirred at room temperature for 1 hour. The reaction solution was extracted with diethyl ether, the organic layer was washed with a saturated aqueous sodium bicarbonate solution, with saturated saline and with distilled water, and then dried over anhydrous magnesium sulfate. The organic layer was then distilled off under reduced pressure to obtain the target substance 4-methylthiofurfural as a crude oil. Sodium borohydride (1.26 g) was gradually added to a methanol solution (70 mL) containing the obtained 4-methylthiofurfural while cooling on ice, and the mixture was stirred for 30 minutes. The reaction solution was distilled under reduced pressure, distilled water was added to the obtained residue, and extraction was performed with dichloromethane. After washing the organic layer with saturated saline and drying over anhydrous magnesium sulfate, the solvent was distilled off under reduced pressure. The obtained residue was subjected to silica gel column chromatography (ethyl acetate:hexane=1:5) to obtain the target substance (4-methylthiofuran-2-yl)methanol (4.40 g) as an oil. The results of structural analysis of the obtained substance were as follows.

¹H-NMR(CDCl₃) δ: 2.35 (3H, s), 4.58 (2H, d, J=6.10 Hz), 6.33 (1H, s), 7.32 (1H, s).

2) Sodium azide (2.98 g) and triphenylphosphine (12.0 g) were added to a DMF solution (60 mL) containing the. (4-methylthiofuran-2-yl)methanol (4.40 g) obtained in 1), after which carbon tetrabromide (15.2 g) was added while cooling on ice and the mixture was stirred at room temperature for 1 hour. The reaction solution was poured into ice water and. extracted with diethyl ether, and the organic layer was washed with saturated saline. After drying the organic layer over anhydrous magnesium sulfate, the solvent was distilled off under reduced pressure. The obtained residue was subjected to silica gel column chromatography (ethyl acetate:hexane=1:5) to obtain the target substance 2-azidomethyl-4-methylthiofuran (3.51 g) as an oil. The results of structural analysis of the obtained substance were as follows.

¹H-NMR(CDCl₃) δ: 2.36 (3H, s), 4.26 (2H, s), 6.37 (1H, s), 7.34 (1H, s).

3) Aluminum lithium hydride (0.34 g) was gradually added to a diethyl ether solution (18 mL) containing the 2-azidomethyl-4-methylthiofuran obtained in 2) (1.52 g) while cooling on ice, and the mixture was stirred for 1 hour. The reaction solution was poured into ice water and the obtained suspension was filtered with celite. The filtrate was extracted with diethyl ether, the organic layer was extracted with 2 N hydrochloric acid, and the extract was rendered alkaline with a 2 N sodium hydroxide aqueous solution and then extracted with diethyl ether. After drying the organic layer over anhydrous magnesium sulfate, the solvent was distilled off under reduced pressure to obtain the target substance (4-methylthiofuran-2-yl)methylamine (0.96 g) as an oil. The results of structural analysis of the obtained substance were as follows.

¹H-NMR(CDCl₃) 67 : 2.34 (3H, s), 3.79 (2H, s), 6.18 (1H, s), 7.26 (1H, s)

4) Trimethylsilyl trifluoromethanesulfonate (2.22 mL) was added to a 1,2-dichloroethane solution (8 mL) containing (4-methylthiofuran-2-yl)methylamine (1.60 g), and the mixture was stirred at room temperature for 1 hour, after which cyanoguanidine (940 mg) was added and the mixture was heated to reflux for 2 hours. The reaction solution was subjected to amine treatment silica gel column chromatography (methanol:chloroform=10:100) to obtain the target substance as an oil (1.10 g). The results of structural analysis of the obtained oily substance were as follows.

¹H-NMR(DMSO-d₆) δ: 2.33 (3H, s), 4.28 (2H, s), 6.39 (1H, s), 6.40-8.31 (6H, m), 7.56 (1H, s); MS(ESI⁺): 228[M+1]⁺; HPLC RT: 4.8 min. (mobile phase: 50% methanol).

The structural formula of the obtained compound is as follows.

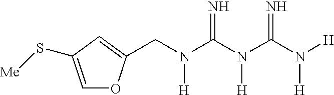

The conditions for amine treatment silica gel column chromatography and LCMS were the same as for Synthesis Example 3, and the HPLC conditions were the same as for Synthesis Example 2 except that the methanol concentration for the mobile phase was as indicated above.

### Synthesis Example 7

### Synthesis of 1-[(5-Methylthiomethylfuran-2-yl)methyll] Biguanide

1) Sodium azide (7.94 g) and triphenylphosphine (32.0 g) were added to a DMF solution (240 mL) of 5-methylthiomethylfuran-2-yl)methanol (12.89 g), after which carbon tetrabromide (40.5 g) was added while cooling on ice and the mixture was stirred at room temperature for 1 hour. The reaction solution was poured into ice water and extracted with diethyl ether and the organic layer was washed with saturated saline and dried over anhydrous magnesium sulfate, after which the solvent was distilled off under reduced pressure. Diethyl ether was added to the obtained residue, the precipitated insoluble portion was filtered off, and the filtrate was distilled under reduced pressure. The obtained residue was subjected to silica gel column chromatography (ethyl acetate:hexane=1:10) to obtain the target substance 2-azidomethyl-5-methylthiomethylfuran (6.01 g) as an oil. The results of structural analysis of the obtained substance were as follows.

¹H-NMR(CDCl₃) δ: 2.10 (3H, s), 3.67 (2H, s), 4.27 (2H, s), 6.16 (1H, d, J=3.13 Hz), 6.28 (1H, d, J=2.97 Hz).

2) Aluminum lithium hydride (1.24 g) was gradually added to a diethyl ether solution (65 mL) containing the 2-azidomethyl-5-methylthiomethylfuran obtained in 1) (6.01 g) while cooling on ice, and the mixture was stirred for 1 hour. The reaction solution was poured into ice water and the obtained suspension was filtered with celite. The filtrate was extracted with diethyl ether, the organic layer was extracted with 2 N hydrochloric acid, and the extract was rendered alkaline with a 2 N sodium hydroxide aqueous solution and then extracted with diethyl ether. After drying the organic layer over anhydrous magnesium sulfate, the solvent was distilled off under reduced pressure to obtain the target substance (5-methylthiomethylfuran-2-yl)methylamine (3.24 g) as an oil. The results of structural analysis of the obtained substance were as follows.

¹H-NMR(CDCl₃) δ: 2.09 (3H, s), 3.66 (2H, s), 3.80 (2H, s), 6.05 (1H, d, J=3.13 Hz), 6.09 (1H, d, J=2.97 Hz).

3) Trimethylsilyl trifluoromethanesulfonate (4.50 mL) was added to a 1,2-dichloroethane solution (23 mL) containing (5-methylthiomethylfuran-2-yl)methylamine (3.24 g), and the mixture was stirred at room temperature for 30 minutes, after which cyanoguanidine (1.90 g) was added and the mixture was heated to reflux for 2 hours. The reaction solution was subjected to amine treatment silica gel column chromatography (methanol:chloroform=10:100) to obtain the target substance as an oil (3.80 g). The results of structural analysis of the obtained oily substance were as follows.

¹H-NMR(DMSO-d₆) δ: 2.03 (3H, s), 3.69 (2H, s), 4.29 (2H, s), 6.20 (1H, d, J=2.80 Hz), 6.23 (1H, d, J=2.80 Hz), 6.40-8.30 (6H, m); MS(ESI⁺): 242[M+1]⁺; HPLC RT: 8.0 min. (mobile phase: 30% methanol).

The structural formula of the obtained compound is as follows.

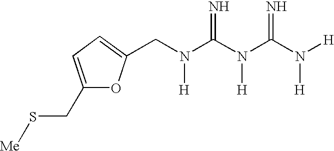

The conditions for amine treatment silica gel column chromatography and LCMS were the same as for Synthesis Example 3, and the HPLC conditions were the same as for Synthesis Example 2 except that the methanol concentration for the mobile phase was as indicated above.

### Synthesis Example 8

### Synthesis of 1-[(3-Methylthiomethylfuran-2-yl)methyl] Biguanide

1) A Vilsmeier reagent prepared with DMF (6.8 mL) and phosphorus oxychloride (8.2 mL) was added dropwise to a DMF solution (80 mL) of 3-methylthiomethylfuran (8.07 g) while cooling on ice, and after stirring the mixture for 1 hour and 45 minutes at room temperature, it was subsequently stirred for 1 hour and 15 minutes in a 45° oil bath. After cooling the reaction solution to room temperature, it was poured into a 2 N sodium hydroxide aqueous solution and extracted with diethyl ether. The organic layer was dried over anhydrous magnesium sulfate and the solvent was distilled off under reduced pressure to obtain the target substance 3-methylthiomethylfurfural as an oil. Sodium borohydride (2.38 g) was gradually added to a methanol solution (120 mL) containing the obtained 3-methylthiomethylfurfural while cooling on ice, and the mixture was stirred for 1 hour. The reaction solution was distilled under reduced pressure, distilled water was added to the obtained residue, and extraction was performed with dichloromethane. After washing the organic layer with saturated saline and drying over anhydrous magnesium sulfate, the solvent was distilled off under reduced pressure to obtain the target substance (3-methylthiomethylfuran-2-yl)methanol (6.40 g) as an oil. The results of structural analysis of the obtained substance were as follows.

¹H-NMR(CDCl₃) δ: 2.05 (3H, s), 3.56 (2H, s), 4.61 (2H, d, J=5.93 Hz), 6.37 (1H, d, J=1.32 Hz), 7.33 (1H, d, J=1.48 Hz).

2) Sodium azide (5.55 g) and triphenylphosphine (22.4 g) were added to a DMF solution (150 mL) containing the (3-methylthiomethylfuran-2-yl)methanol (9.00 g) obtained in 1), after which carbon tetrabromide (28.3 g) was added while cooling on ice and the mixture was stirred at room temperature for 1 hour. The reaction solution was poured into ice water and extracted with diethyl ether, and the organic layer was washed with saturated saline. After drying the organic layer over anhydrous magnesium sulfate, the solvent was distilled off under reduced pressure. Diethyl ether was added to the obtained residue, the precipitated insoluble portion was filtered off, and the filtrate was distilled under reduced pressure. The obtained residue was subjected to silica gel column chromatography (ethyl acetate:hexane=1:5) to obtain the target substance 2-azidomethyl-3-methylthiomethylfuran (9.03 g) as an oil. The results of structural analysis of the obtained substance were as follows.

¹H-NMR(CDCl₃) δ: 2.04 (3H, s), 3.52 (2H, s), 4.32 (2H, s), 6.41 (1H, d, J=1.65 Hz), 7.38 (1H, d, J=1.82 Hz).

3) Aluminum lithium hydride (2.20 g) was gradually added to a diethyl ether solution (290 mL) containing the 2-azidomethyl-3-methylthiomethylfuran obtained in 2) (10.5 g) while cooling on ice, and the mixture was stirred for 1 hour. The reaction solution was poured into ice water and the obtained suspension was filtered with celite. The filtrate was extracted with diethyl ether, the organic layer was extracted with 2 N hydrochloric acid, and the extract was rendered alkaline with a 2 N sodium hydroxide aqueous solution and then extracted with diethyl ether. After drying the organic layer over anhydrous magnesium sulfate, the solvent was distilled off under reduced pressure to obtain the target substance (3-methylthiomethylfuran-2-yl)methylamine (2.20 g) as an oil. The results of structural analysis of the obtained substance were as follows.

¹H-NMR(CDCl3) δ: 2.03 (3H, s), 3.51 (2H, s), 3.79 (2H, s), 6.33 (1H, d, J=1.49 Hz), 7.29 (1H, d, J=1.65 Hz).

4) Trimethylsilyl trifluoromethanesulfonate (2.78 mL) was added to a 1,2-dichloroethane solution (10 mL) containing (3-methylthiomethylfuran-2-yl)methylamine (2.20 g), and the mixture was stirred at room temperature for 40 minutes, after which cyanoguanidine (1.18 g) was added and the mixture was heated to reflux for 1 hour. The reaction solution was subjected to amine treatment silica gel column chromatography (methanol:chloroform=10:100) to obtain the target substance as an oil (1.62 g). The results of structural analysis of the obtained oily substance were as follows.

¹H-NMR(DMSO-d₆) δ: 1.97 (3H, s), 3.56 (2H, s), 4.32 (2H, s), 6.41 (1H, d, J=1.65 Hz), 7.56 (1H, d, J=1.83 Hz), 6.40-8.30 (6H, m); MS(ESI⁺): 242[M+1]⁺; HPLC RT: 5.7 min. (mobile phase: 30% methanol).

The structural formula of the obtained compound is as follows.

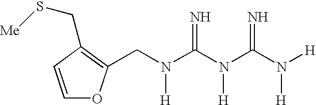

The conditions for amine treatment silica gel column chromatography and LCMS were the same as for Synthesis Example 3, and the HPLC conditions were the same as for Synthesis Example 2 except that the methanol concentration for the mobile phase was as indicated above.

### Examples 1-5 and Comparative Examples 1-5

### Hypoglycemic Test by Oral Administration Using KKAy Mice

A group of six 11-week-old male mice (KKAy/Ta) was used for the test. Blood was sampled from the tail for measurement of the blood glucose levels before treatment as a control. After sampling, furfuryl biguanide was dissolved in a 0.5% CMC-Na (sodium carboxymethyl cellulose) solution to a suitable concentration and orally administered at 10 mL/kg with the dosages shown in Table 1 (Examples 1-5). For comparison, metformin was also orally administered to mice in the doses shown in Table 1 (Comparative Examples 1-5). As a control there were prepared mice which had been orally administered the solvent alone. Blood was sampled from the tail to measure the blood glucose levels 1, 2, 4 and 6 hours after administration of the drug, and the blood glucose reduction rates were calculated by the formula given above. The blood glucose levels were measured using a Glucose CIT-Test Wako (Wako Pure Chemical Industries, Ltd.). The test results are shown in Table 1.

TABLE 1

| | | | Blood |
|---|---|---|---|
| | | | glucose |
| | | Dosage | reduction |
| | Compound name | (mg/kg) | rate (%) |
| Example 1 | Furfuryl | 25.0 | 8.7 |
| | biguanide | | |
| Example 2 | Furfuryl | 50.0 | 15.2 |
| | biguanide | | |
| Example 3 | Furfuryl | 100.0 | 29.4 |
| | biguanide | | |
| Example 4 | Furfuryl | 200.0 | 43.9 |
| | biguanide | | |
| Example 5 | Furfuryl | 300.0 | 48.0 |
| | biguanide | | |
| Comp. Ex. 1 | Metformin | 150.0 | 11.6 |
| Comp. Ex. 2 | Metformin | 300.0 | 6.4 |
| Comp. Ex. 3 | Metformin | 600.0 | 21.8 |
| Comp. Ex. 4 | Metformin | 900.0 | 32.7 |
| Comp. Ex. 5 | Metformin | 1350.0 | 46.4 |

### Examples 6-22 and Comparative Examples 6-18

### Oral Glucose Tolerance Test

Eleven- to seventeen-week-old female mice (C57BLKS/J-m+/+Lepr<db>(db/db)) were starved for 18-24 hours, and a group of six mice was used for the test. Blood was sampled from the tail for measurement of the blood glucose levels and blood lactic acid levels before treatment. After sampling, the compounds listed in Tables 2 to 4 (Examples 6-22) were dissolved in phosphate-buffered physiological saline to give the dosages also listed in Tables 2 to 4, and were subcutaneously administered to the mice at a dose of 5 ml/kg. For comparison, metformin was also subcutaneously administered to mice in the doses shown in Table 2 (Comparative Examples 6-19). As a control there were prepared mice which had been subcutaneously administered the solvent alone.

Glucose was then administered orally at a dose of 3 g/6 ml/kg at 30 minutes after administration of the compound or solvent as an oral glucose tolerance test. Blood was sampled from the tail for measurement of the blood glucose levels and blood lactic acid levels 30 minutes, 1 hour and 2 hours after glucose administration, and the blood glucose reduction rates and blood lactic acid level increase rates were calculated by the formula given above. The blood glucose levels were measured using a New Blood Sugar Test (Roche Diagnostics) or a Glucose CII-Test Wako (Wako Pure Chemical Industries, Ltd.). The blood lactic acid levels were measured using an “Asuka Sigma” (Sigma Diagnostics). The test results are shown in Tables 2 to 4.

TABLE 2

| | | | | Blood |
|---|---|---|---|---|
| | | | Blood | lactic acid |
| | | | glucose | level |
| | Compound | Dosage | reduction | increase |
| | name | (mg/kg) | rate (%) | rate (%) |
| Example 6 | Furfuryl | 20.0 | 46.9 | 7.0 |
| | biguanide | | | |
| Example 7 | Furfuryl | 40.0 | 52.3 | 16.7 |
| | biguanide | | | |
| Example 8 | Furfuryl | 75.0 | 60.6 | 22.0 |
| | biguanide | | | |
| Example 9 | Furfuryl | 80.0 | 68.7 | 11.2 |
| | biguanide | | | |
| Comp. Ex. 6 | Metformin | 100 | 19.4 | 7.6 |
| Comp. Ex. 7 | Metformin | 100 | 17.3 | 24.2 |
| Comp. Ex. 8 | Metformin | 150 | 28.1 | 33.5 |
| Comp. Ex. 9 | Metformin | 150 | 36.1 | 16.6 |
| Comp. Ex. 10 | Metformin | 150 | 22.0 | 18.4 |
| Comp. Ex. 11 | Metformin | 150 | 51.9 | 18.2 |
| Comp. Ex. 12 | Metformin | 150 | 23.9 | 35.6 |
| Comp. Ex. 13 | Metformin | 200 | 37.5 | 36.8 |
| Comp. Ex. 14 | Metformin | 200 | 37.7 | 42.7 |
| Comp. Ex. 15 | Metformin | 300 | 74.1 | 133.7 |
| Comp. Ex. 16 | Metformin | 300 | 72.7 | 155.9 |
| Comp. Ex. 17 | Metformin | 300 | 93.4 | 138.9 |
| Comp. Ex. 18 | Metformin | 300 | 92.3 | 145.9 |

TABLE 3

| | | | Blood |
|---|---|---|---|
| | | | glucose |
| | | Dosage | reduction |
| | Compound name | (mg/kg) | rate (%) |
| Example 10 | 1-(5-methylfurfuryl) | 100 | 86.6 |
| | biguanide | | |
| Example 11 | 1-[(5-ethylfuran-2- | 75 | 71.3 |
| | yl)methyl] biguanide | | |
| Example 12 | 1-[(5-tert-butylfuran- | 75 | 62.1 |
| | 2-yl)methyl] biguanide | | |
| Example 13 | 1-[(4,5-dimethylfuran- | 75 | 52.0 |
| | 2-yl)methyl] biguanide | | |
| Example 14 | 1-[(4-methylthiofuran- | 75 | 44.1 |
| | 2-yl)methyl] biguanide | | |
| Example 15 | 1-[(4-methylthiofuran- | 100 | 47.5 |
| | 2-yl)methyl] biguanide | | |
| Example 16 | 1-[(4-methylthiofuran- | 150 | 58.1 |
| | 2-yl)methyl] biguanide | | |
| Example 17 | 1-[(4-methylthiofuran- | 200 | 73.9 |
| | 2-yl)methyl] biguanide | | |
| Example 18 | 1-[(5-methylthiomethylfuran- | 75 | 46.0 |
| | 2-yl)methyl] biguanide | | |
| Example 19 | 1-[(3-methylthiomethylfuran- | 150 | 47.3 |
| | 2-yl)methyl] biguanide | | |

TABLE 4

| | | | | Blood |
|---|---|---|---|---|
| | | | Blood | lactic acid |
| | | | glucose | level |
| | | Dosage | reduction | increase |
| | Compound name | (mg/kg) | rate (%) | rate (%) |
| Example 20 | 1-(5-methylfurfuryl) | 25 | 24.7 | 3.9 |
| | biguanide | | | |
| Example 21 | 1-(5-methylfurfuryl) | 50 | 46.1 | 1.3 |
| | biguanide | | | |
| Example 22 | 1-(5-methylfurfuryl) | 75 | 54.7 | 14.7 |
| | biguanide | | | |

As clearly shown by the results in Table 1, administration of a biguanide derivative of the invention represented by general formula (1) above, or its salt, was confirmed to adequately suppress blood glucose level increase by a notable insulin sensitivity-enhancing effect. Also, as clearly shown by the results in Tables 2 to 4, administration of a biguanide derivative of the invention represented by general formula (1) above or its salt was also confirmed to extremely minimize increase in blood lactic acid levels while exhibiting a notable hypoglycemic effect.

As explained above, the present invention provides pharmaceutical preparations for treatment of type II diabetes which have effects of satisfactorily suppressing blood glucose level increase by enhancing insulin sensitivity, and of adequately lowering blood glucose levels preferably while sufficiently suppressing increase in blood lactic acid levels.

Consequently, according to the invention it is possible to provide pharmaceutical preparations for treatment and prophylactic agents of type II diabetes which have adequate insulin sensitivity-enhancing effects with low risk of eliciting side-effects such as lactic acidosis, as well as therapeutic and prophylactic methods using the preparations. According to the invention it is also possible to provide prophylactic agents and prophylactic methods using them, which are effective for prevention of large artery obstruction including myocardial infarction and cerebral apoplexy.

What is claimed is:

1. A pharmaceutical preparation for type II diabetes comprising as an active ingredient thereof a biguanide derivative represented by the following general formula (1): 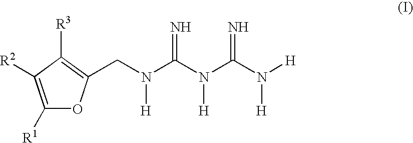
(where R¹, R² and R³ are the same or different and each represents one selected from the group consisting of hydrogen, optionally substituted lower alkyl groups and optionally substituted lower alkylthio groups), or a salt thereof.

2. A pharmaceutical preparation for type II diabetes according to claim 1, wherein the biguanide derivative represented by general formula (1) above is furfuryl biguanide.

3. A pharmaceutical preparation for type II diabetes according to claim 1, which is intended as a treatment for suppressing blood glucose level increase by enhancing insulin sensitivity.

4. A pharmaceutical preparation for type II diabetes according to claim 1, which has an effect of lowering blood glucose levels essentially without raising blood lactic acid levels.

5. A pharmaceutical preparation for type II diabetes according to claim 1, wherein the target disease is one selected from the group consisting of diabetes accompanied by lactic acidosis anamnesis, diabetes accompanied by impaired renal function, diabetes accompanied by impaired hepatic function, diabetes accompanied by impaired cardiovascular, diabetes accompanied by impaired pulmonary function, diabetes accompanied by tendency to hypoxemia, diabetes in individuals with excessive alcohol intake, diabetes accompanied by gastrointestinal injury, and diabetes in individuals of advanced age.

6. A method for treatment of type II diabetes which comprises administering a biguanide derivative represented by the following general formula (1): 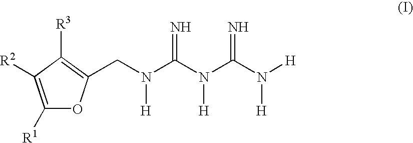
(where R¹, R² and R³ are the same or different and each represents one selected from the group consisting of hydrogen, optionally substituted lower alkyl groups and optionally substituted lower alkylthio groups), or a salt thereof.

7. A method for treatment of type II diabetes according to claim 6, wherein the biguanide derivative represented by general formula (1) above is furfuryl biguanide.

8. A method for treatment of type II diabetes according to claim 6, which is a method of treatment for suppressing blood glucose level increase by enhancing insulin sensitivity.

9. A method for treatment of type II diabetes according to claim 6, whereby blood glucose levels are lowered essentially without raising blood lactic acid levels.

10. A method for treatment of type II diabetes according to claim 6, wherein the target disease is one selected from the group consisting of diabetes accompanied by lactic acidosis anamnesis, diabetes accompanied by impaired renal function, diabetes accompanied by impaired hepatic function, diabetes accompanied by impaired cardiovascular, diabetes accompanied by impaired pulmonary function, diabetes accompanied by tendency to hypoxemia, diabetes in individuals with excessive alcohol intake, diabetes accompanied by gastrointestinal injury, and diabetes in individuals of advanced age.

11. A prophylactic agent for type II diabetes comprising as an active ingredient thereof a biguanide derivative represented by the following general formula (1): 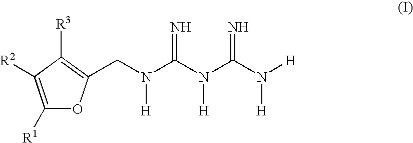
(where R¹, R² and R³ are the same or different and each represents one selected from the group consisting of hydrogen, optionally substituted lower alkyl groups and optionally substituted lower alkylthio groups), or a salt thereof.

12. A method for prevention of type II diabetes which comprises administering a biguanide derivative represented by the following general formula (1): 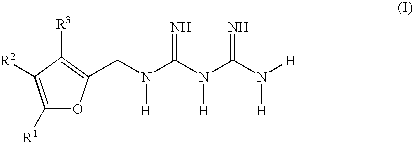
(where R¹, R² and R³ are the same or different and each represents one selected from the group consisting of hydrogen, optionally substituted lower alkyl groups and optionally substituted lower alkylthio groups), or a salt thereof.

13. A prophylactic agent for large artery obstruction comprising as an active ingredient thereof a biguanide derivative represented by the following general formula (1): 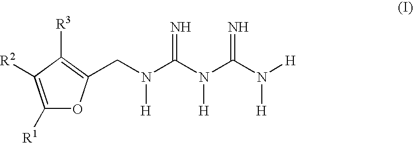
(where R¹, R² and R³ are the same or different and each represents one selected from the group consisting of hydrogen, optionally substituted lower alkyl groups and optionally substituted lower alkylthio groups), or a salt thereof.

14. A method for prevention of large artery obstruction which comprises administering a biguanide derivative represented by the following general formula (1): 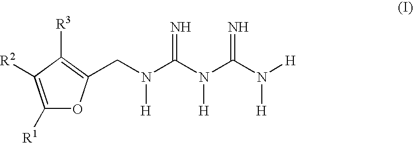
(where R¹, R² and R³ are the same or different and each represents one selected from the group consisting of hydrogen, optionally substituted lower alkyl groups and optionally substituted lower alkylthio groups), or a salt thereof.

15. A biguanide derivative represented by the following general formula (1): 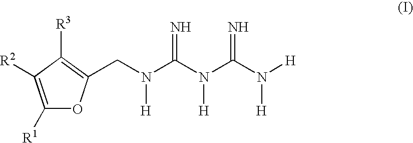
(where R¹, R² and R³ are the same or different and each represents one selected from the group consisting of hydrogen, optionally substituted lower alkyl groups and optionally substituted lower alkylthio groups, except for furfuryl biguanide wherein R¹, R² and R³ are all hydrogen and 1-[ (5-methylfuran-2-yl)methyl] biguanide wherein R¹ is methyl and R² and R3 are both hydrogen).
